(19) United States
(12) Patent Application Publication
(10) Pub. No.: US 2008/0021364 A1
(43) Pub. Date: Jan. 24, 2008

(54) FLUIDIC DEVICE

(75) Inventor: Kuo-Yao Weng, Hsin Chu (TW)

Correspondence Address: FISH & RICHARDSON PC, P.O. BOX 1022, MINNEAPOLIS, MN, 55440-1022, US

(73) Assignee: Industrial Technology Research Institute, Hsin Chu (TW)

(21) Appl. No.: 11/612,869

(22) Filed: Dec. 19, 2006

Related U.S. Application Data
(60) Provisional application No. 60/831,285, filed on Jul. 17, 2006.

(51) Int. Cl. A61B 5/15

(52) U.S. Cl. 604/4.01; 604/6.01; 604/6.04

(57) ABSTRACT

A fluidic device that includes a channel and a container that generates a pressure difference in the channel when the container is broken. The container includes a brittle material. The first container (a) defines a space within the first container having a gas pressure that is different from the gas pressure outside of the first container, or (b) includes a first material that is separated from a second material prior to the breaking of the first container. The first and second materials are selected to generate gas upon interaction of the first and second materials.

### CROSS REFERENCE TO RELATED APPLICATIONS

The application claims priority to U.S. provisional application Ser. No. 60/831,285, filed Jul. 17, 2006. This application is related to concurrently filed U.S. patent applications entitled “Fluidic Device” (identified as Attorney Docket 05896-006002), and “Fluidic Device” (Attorney Docket 05896-006003). The above applications are all incorporated by reference.

### BACKGROUND OF THE INVENTION

The description relates to fluidic devices.

Many types of testing devices can be used in detecting the presence of compounds or analyzing bio-chemical reactions. For example, lateral flow assays can be performed using a lateral flow membrane having one or more test lines along its length. A fluid with dissolved reagents travels from one end of the membrane to the test lines by electro osmosis. A reader detects whether reaction occurred at the test lines, which indicate the presence or absence of certain particles in the reagents. As another example, a device with an array of micro capillaries can be used to control the flow of fluids in immunoassay processes. Reagents are positioned at various locations along the lengths of the micro capillaries so that as fluids flow in the micro capillaries due to capillary force, the fluids come into contact with the reagents. A reader monitors the sites where the reagents are located to determine whether reactions have occurred. As yet another example, micro fluidic chips can be used to perform assays by controlling the flow of fluids through various channels and chambers. The micro fluidic chips are used with an external power supply and/or pump that provide the driving force for moving the fluids.

### SUMMARY

In one aspect, in general, a method includes controlling a flow of a fluid in a channel, including breaking a first container to generate a pressure difference in the channel to cause the fluid to move in the channel, the first container being made of a brittle material. The first container (a) defines a space within the first container having a gas pressure that is different from the gas pressure outside of the first container, or (b) includes a first material that is separated from a second material prior to the breaking of the first container, in which the first and second materials are selected to generate gas upon interaction of the first and second materials.

Implementations of the method can include one or more of the following features. In some examples, the space in the first container can have a pressure higher than the pressure outside of the first container. Controlling the flow of the fluid can include pushing the fluid in the channel away from the broken first container. In some examples, the space in the first container can have a pressure lower than the pressure outside of the first container. In some examples, controlling the flow of the fluid can include attracting the fluid in the channel towards the broken first container. The fluid can be, for example, blood. Controlling the flow of the blood can include passing the blood through a filter to block blood cells and to allow blood plasma to pass the filter and enter the channel. The method can include performing a colorimetric assay as the fluid flows in the channel. The method can include a second container that (a) defines a space within the second container having a gas pressure that is higher than the gas pressure outside of the second container, or (b) includes a third material that is separated from a fourth material prior to the breaking of the second container, the third and fourth materials selected to generate gas upon interaction of the third and fourth materials. In some examples, controlling the flow of the fluid can include pushing the fluid in the channel away from the second container. Controlling the flow of the fluid can include preventing movement of additional fluid through the channel in a certain direction by using a fluid absorbing material that expands in volume upon absorption of a portion of the fluid.

In another aspect, in general, a fluidic device includes a channel, and a first container that generates a pressure difference in the channel when the first container is broken, in which the first container is made of a brittle material. The first container (a) defines a space within the first container having a gas pressure that is different from the gas pressure outside of the first container, or (b) includes a first material that is separated from a second material prior to the breaking of the first container, in which the first and second materials are selected to generate gas upon interaction of the first and second materials.

Implementations of the fluidic device can include one or more of the following features. The fluidic device can include a second container that (a) defines a space within the second container having a gas pressure that is different from the gas pressure outside of the second container, or (b) includes a third material that is separated from a fourth material prior to the breaking of the second container, in which the third and fourth, materials are selected to generate gas upon interaction of the third and fourth materials. In some examples, the space in the first container can have a pressure higher than the pressure outside of the first container, and the space in the second container can have a pressure lower than the pressure outside of the second container. In some examples, the space in the first container can have a pressure lower than the pressure outside of the first closed container, and the second container can include a first material that is separated from a second material prior to the breaking of the second container, the first and second materials selected to generate gas upon interaction of the first and second materials. The fluidic device can include a filter membrane to block blood cells and to allow blood plasma to pass. The brittle material can include at least one of quartz, glass, ceramic, plastic, and a composite of two or more of quartz, glass, ceramic, and plastic. The channel can be defined by a wall made of a flexible material. The first container can include a material that generates gas when heated. The first container can include a material that sublimes from a solid state to a gas state when heated.

In another aspect, in general, a method includes providing a plurality of pipettes to enable sampling of predetermined amounts of fluids, each pipette including a channel, and a container that generates a pressure difference in the channel when the container is broken. The container is made of a brittle material and defines a space within the container having a gas pressure that is less than the gas pressure outside of the container. Breaking the container generates a predetermined amount of pressure difference in the channel to cause a predetermined amount of fluid to be drawn into the channel.

Implementations of the fluidic device can include one or more of the following features. Each pipette includes a second container that generates a pressure difference in the channel when the second container is broken, the second container (a) defining a space within the second container having a gas pressure that is higher than the gas pressure in the channel, or (b) including a first material that is separated from a second material prior to the breaking of the second container, the first and second materials selected to generate gas upon interaction of the first and second materials. The brittle material includes at least one of quartz, glass, ceramic, plastic, and a composite of two or more of quartz, glass, ceramic, and plastic.

### DESCRIPTION OF DRAWINGS

FIGS. 1A and 1B are schematic diagrams of a vacuum pump.

FIGS. 2A and 2B are schematic diagrams of a gas pump.

FIGS. 3A and 3B are schematic diagrams of a gas pump.

FIG. 4A is a schematic diagram of a gas pump.

FIG. 4B is a table of materials.

FIGS. 5A and 5B are schematic diagrams of a broken-open valve.

FIGS. 6A, 6B, 7A, 7B, and 8A to 8C are schematic diagrams of self-close valves.

FIGS. 9A to 9C are schematic diagrams of an on-off-on valve.

FIGS. 10A to 10C are schematic diagrams of an off-on-off valve.

FIGS. 11A to 11D are schematic diagrams of an on-off-on-off valve.

FIG. 12 is a schematic diagram of a metering pipette.

FIG. 13 is a schematic diagram of a metering pipette.

FIGS. 14A to 14C are schematic diagrams of a metering pipette.

FIGS. 15A and 15B are schematic diagrams of a metering device.

FIGS. 16A and 16B are schematic diagrams of a metering device.

FIGS. 17A to 17C are schematic diagrams of a device for use in a two-step assay.

FIGS. 18A to 18C are schematic diagrams of a device for use in a two-step assay.

FIGS. 19A to 19C are schematic diagrams of a device for use in a three-step assay.

FIG. 20 is a schematic diagram of a module for use in a multiplex analyte assay.

FIGS. 21A and 21B show a metering pipette being used to sample blood from a patient.

FIGS. 22A and 22B are schematic diagrams of a device for performing rapid reaction colorimetric assay.

FIGS. 23A and 23B are schematic diagrams of a device for sampling a filtered fluid.

FIGS. 24A to 24C are schematic diagrams of a device for performing a slow colorimetric assay.

FIGS. 25A to 25C are schematic diagrams of vacuum pumps.

FIGS. 26A and 26B are schematic diagrams of vacuum pumps.

FIGS. 27A to 27C are schematic diagrams of self-close valves.

FIGS. 28A and 28B are schematic diagrams of a broken open valve.

FIG. 28C is a cross section of a glass capillary.

FIGS. 29A and 29B are a diagram and a photograph, respectively, of a device for performing an immunoassay.

FIGS. 30A to 30C are diagrams showing steps for performing the immunoassay using the device of FIG. 29A.

FIG. 31 is a photograph of a device for performing an immunoassay.

### DESCRIPTION

A fluidic device for performing assays can include control components such as vacuum pumps, gas pumps, “broken open valves,” and “self-close valves” for controlling the flow of fluids in the fluidic device. The vacuum pump can be used to pull a fluid in a specific direction in a channel, and the gas pump can be used to push a fluid in a specific direction in a channel. The broken open valve can be used to connect two separate regions at the control of a user, and the self-close valve can be used to automatically seal off a channel after passage of a fluid. The vacuum pumps, gas pumps, broken open valves, and self close valves can be made small so that the fluidic device can be made small and portable.

In the following description, tire individual control components will be introduced first, followed by a description of how the control components can be combined to construct modular units for controlling fluids in fluidic devices. Afterwards, how biological assays can be performed using the fluidic devices will be described.

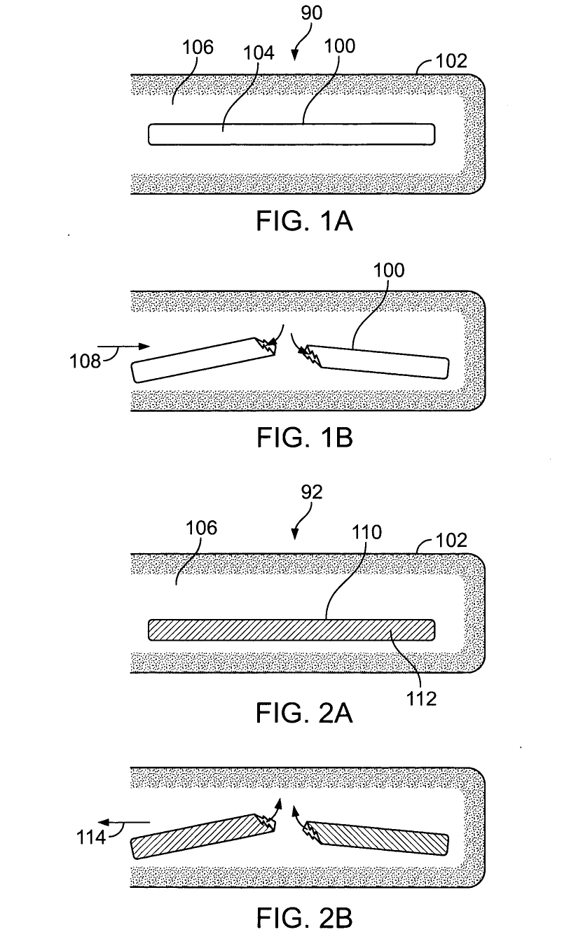

Referring to FIG. 1A, a vacuum pump 90 can be constructed by placing a container 100 in a channel 106 (or chamber) defined by a material 102. The container 100 encloses a region 104 that is vacuum or has a low gas pressure as compared to the gas pressure in the channel 106.

Referring to FIG. 1B, the container 100 can he, e.g., a glass capillary, that breaks upon application of an external force. When the container 100 breaks, gas in the channel 106 flows into the vacuum region 104, reducing the pressure in the region 106. This produces a suction force that can be used to pull a fluid in a direction 108 towards the region 106.

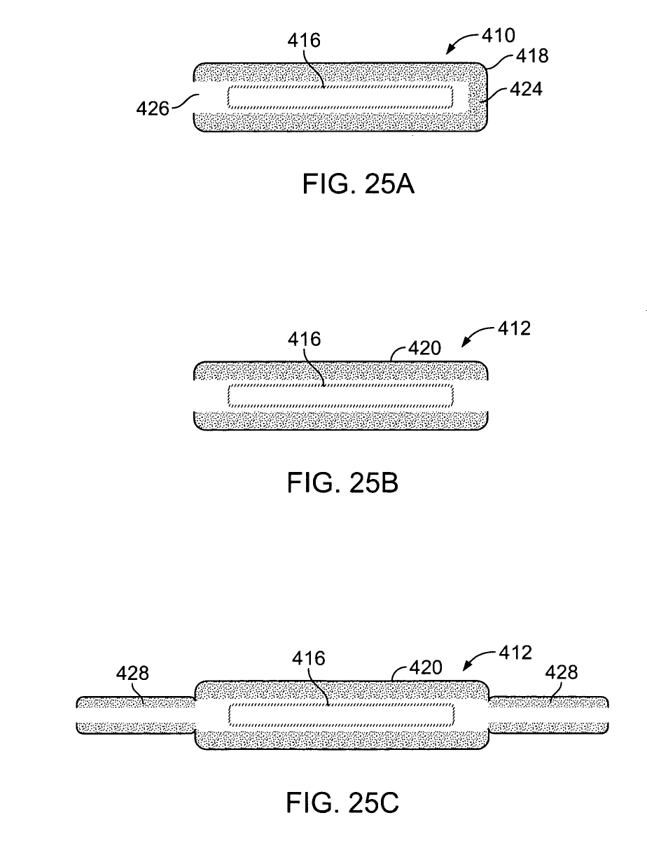

FIGS. 25A to 25C show examples of vacuum pumps using glass capillaries placed in rubber tubes. FIG. 25A shows a cross section of a gas pump 410 having a vacuum glass capillary 416 placed in a rubber tube 418, where the tube 418 has a closed end 424 and an open end 426. FIG. 25B shows a cross section of a gas pump 412 that is similar to the gas pump 410 except that the gas pump 412 has a rubber tube 420 with two open ends. FIG. 25C shows the gas pump 412 connected to two rubber tubes 428, where die rubber tube 420 has a larger inner diameter (to accommodate the glass capillary 416) than the rubber tubes 428.

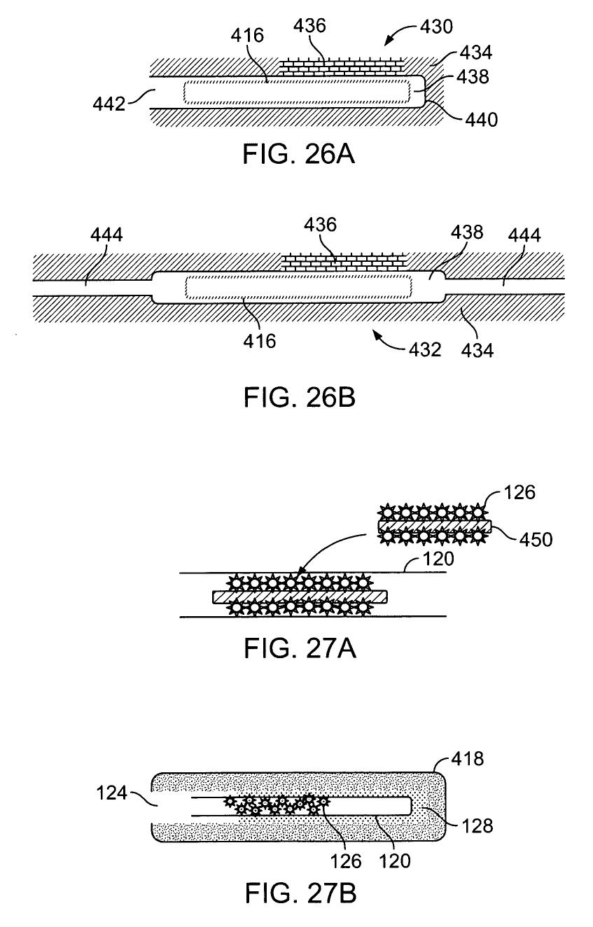

FIGS. 26A and 26B show examples of vacuum pumps using glass capillaries placed in planar fluidic channels. FIG. 26A shows a cross section of a vacuum pump 430 having a vacuum glass capillary 416 placed in a fluidic channel 438 defined by a planar substrate 434. The fluidic channel 438 has a closed end 440 and an open end 442. The planar substrate 434 may be made of a rigid material. An elastic layer 436 is embedded in the substrate 434 at a location adjacent to the capillary 416 to allow a user to apply an external force through the elastic layer to break the capillary 416.

FIG. 26B shows a cross section of a vacuum pump 432 that is similar to the vacuum pump 430 except that the fluidic channel 438 is connected to two fluidic channels 444 having smaller cross sections.

A vacuum glass capillary can be made by heating one end of a glass capillary to melt the glass to form a first closed end. A vacuum pump is used to pump air out of the glass capillary through the open end. The glass capillary is heated at a location at a distance from the first closed end. The heat softens the glass, which can be pinched or twisted to form a second closed end.

Referring to FIG. 2A, a gas pump 92 can be constructed by placing a container 110 in a channel 106 (or chamber) defined by a material 102. The container 110 encloses a region 112 that has a higher gas pressure compared to the gas pressure in the channel 106 outside of the container 110.

Referring to FIG. 2B, the container 110 can be, e.g., a glass capillary, that breaks upon application of an external force. When the container 110 breaks, gas originally inside the container 110 flows out of the container 110, increasing the pressure in the region 106. This produces a force that can be used to push a fluid in a direction 114 away from the region 106.

In this description, the term “vacuum pump” will be used to refer generally to a device that generates a pull force that can be used to pull a fluid towards the device, and the term “gas pump” will be used to refer generally to a device that generates a push force that can be used to push a fluid away from the device.

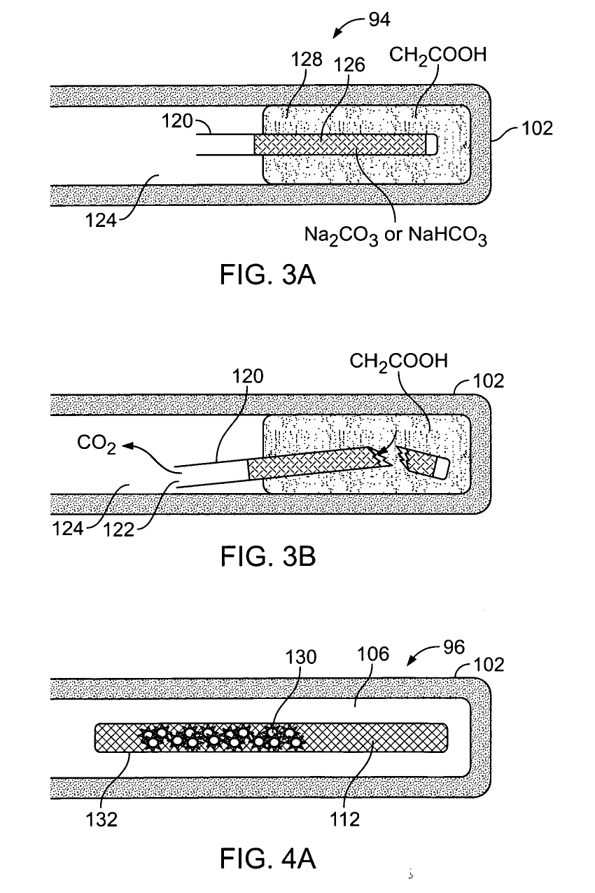

There are alternative ways to construct a gas pump. For example, referring to FIG. 3A, a gas pump 94 can be fabricated by placing a glass capillary 120 that is partially filled with a first material 126 in a channel 124 (or chamber) that contains a second material 128. The first and second materials 126 and 128 are selected so that when they intermix, the materials 126 and 128 will interact and generate one or more gases. For example, the first material 126 can be disodium carbonate (Na₂CO₃) and/or sodium hydrogen carbonate (NaHCO₃), and the second material 128 can be ethanoic acid (CH₂COOH).

Referring to FIG. 3B, when an external force is applied to break the glass capillary 120, the first and second materials 126 and 128 interact and generate a gas. In this example, the gas is carbon dioxide (CO2). The chemical reactions that occur are:

Na₂CO₃+2 CH₂COOH→2 NaCOOCH₂+H₂O+CO₂

NaHCO₃+CH₂COOH→NaCOOCH₂+H₂O+CO₂

### The carbon dioxide increases the pressure in the channel 124, generating a force that can be used to push a fluid away from the broken capillary 120.

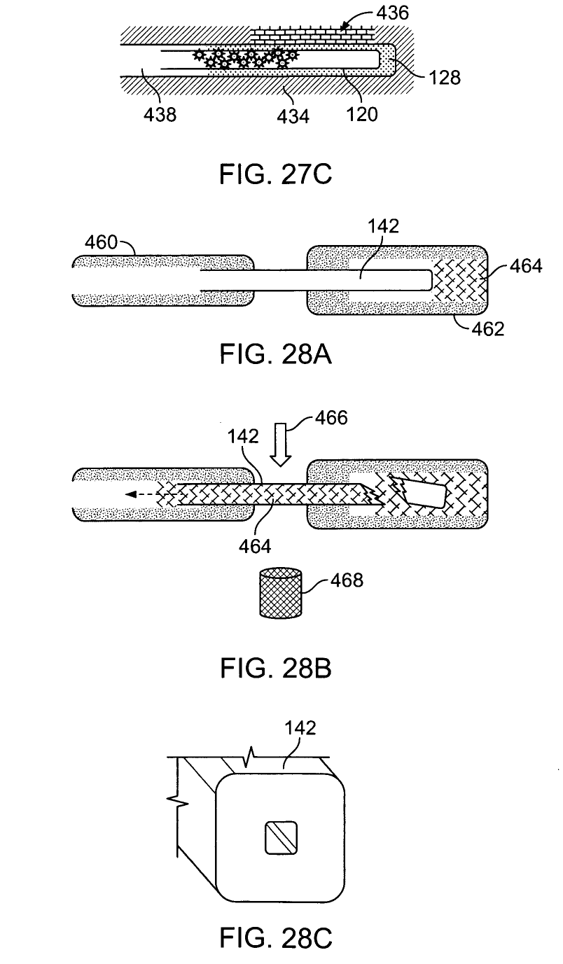

The first material 126 can be filled directly into the capillary 120. Referring to FIG. 27A, the first material 126 can also be attached to a wire 450, then the wire 450 along with the coated material 126 is placed inside the capillary 120. FIG. 27B shows an example in which the glass capillary 120 is placed in a channel 124 within a rubber tube 418. The channel 124 contains a second material 128 that can interact with the first material 126 when the glass capillary 120 is broken. FIG. 27C shows an example in which the glass capillary 120 is placed in a fluidic channel 438 within a planar device substrate 434. An elastic layer 436 is embedded in the substrate 434 at a location adjacent to the capillary 120 to allow a user to apply an external force through the elastic layer 436 to break the capillary 120.

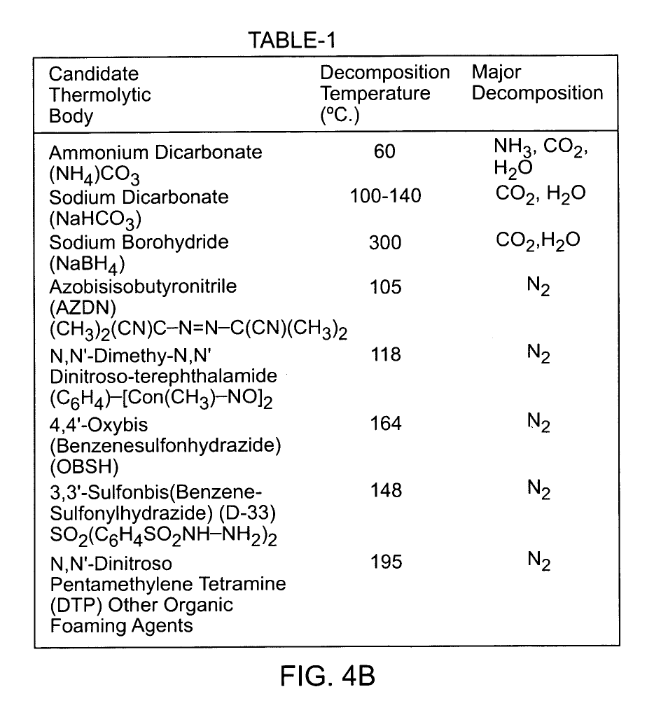

Referring to FIG. 4A, a gas pump 96 can be fabricated by placing a compound 130 in a glass capillary 132, sealing the capillary 132, heating the capillary 132, cooling the capillary 132, and placing the capillary 132 in a channel 106 (or chamber). The compound 130 is selected to be a material that generates a gas after being heated. When the capillary 332 is heated and cooled, the gas generated from the compound 130 increases the gas pressure inside the capillary 132, as compared to the gas pressure outside of the capillary 132.

Examples of the compound 130 include sodium dicarbonate (NaHCO₃) and calcium carbonate (CaCO₃). These compounds generate carbon dioxide when heated:

NaHCO₃→NaOH+CO₂

CaCO₃→CaO+CO₂

The compound 130 can also include sodium azide, NaN₃, which generates N₂ gas by using the thermal decomposition reaction:

2NaN₃→2Na+3N₂.

Sublimation materials that change from solid form to gas form (e.g. dry ice that turns into CO₂) can also be used. Other materials that generate gas when heated are listed in Table 1 of FIG. 4B.

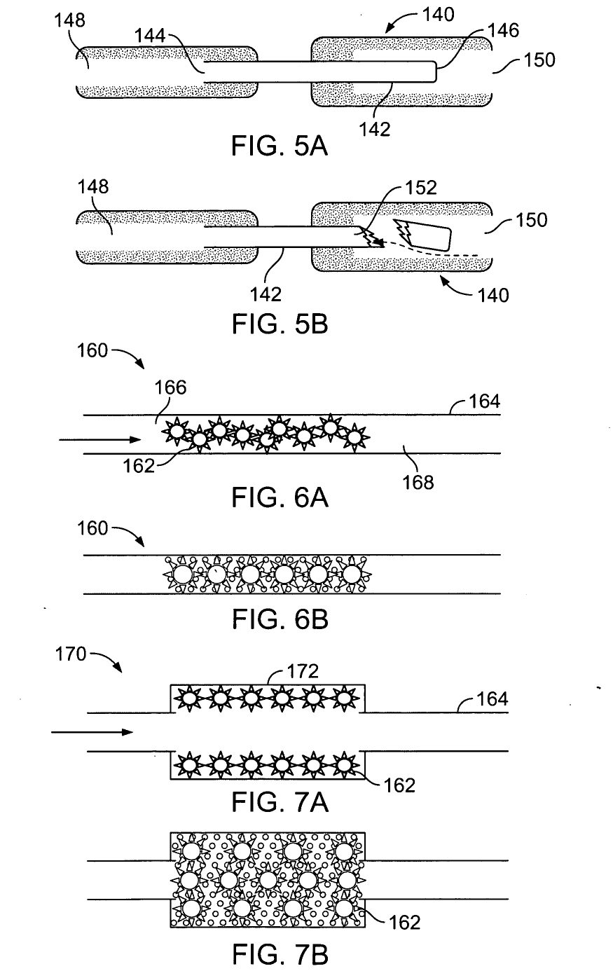

Referring to FIG. 5A, a broken open valve 140 can be fabricated by placing a glass capillary 142 between a first channel 148 and a second channel 150. The glass capillary 142 has an open end 144 that is positioned in the first channel 148, and a closed end 146 that is positioned in the second channel 150. When the glass capillary 142 is intact, fluids cannot flow between the first and second channels 148 and 150. This is referred to as the “closed” state of the broken open valve 140.

Referring to FIG. 5B, when an external force is applied to break the glass capillary 142, a passage 152 is formed that connects the channels 148 and 150. This is referred to as the “open” state of the broken open valve 140. The broken open valve 140 is useful in allowing two fluids (or a fluid and a solid) to be separated initially, then interact at a time controlled by the user.

FIGS. 28A and 28B show an example of using a broken-open valve to construct a low cost device for performing an assay in which a fluid is irradiated with ultra-violet (UV) light. A glass capillary 142 connects two plastic channels 460 and 462. Initially, a reactant 464 is contained in the first plastic channel 462. Upon breaking the glass capillary 142, the reactant 464 flows through the glass capillary 142 to the second plastic channel 460. As shown in FIG. 28B, a UV light source 466 irradiates the reactant 464 as it flows through the glass capillary 142. A detector 468 detects the UV light that passes the reactant 464. The spectrum of the UV light detected by the detector 468 is useful in determining the compounds in the reactant 464.

FIG. 28C shows a cross section of a glass capillary having square shaped inner and outer perimeters. The square shaped inner and outer perimeters allow the UV light to pass the glass capillary in a direction that is perpendicular to the surface of the glass capillary. This allows more UV light to reach the fluid in the glass capillary, as compared to a capillary having a circular cross section that may cause the incident UV light to be reflected or redirected in directions away from the fluid.

Referring to FIGS. 6A and 6B, a self-close valve 160 can be constructed by placing superabsorbent polymers (SAP) 162 in a channel 164. Initially, the SAP 162 has a smaller volume and allows fluids to flow between a first region 166 and a second region 168 in the channel 164 (FIG. 6A). This is referred to as the “open” state of the self-close valve. When a fluid flows past the SAP 162, the SAP absorbs a portion of the fluid and expands in volume, blocking the channel 164 (FIG. 6B), preventing additional fluid from flowing between the first region 166 and the second region 168. This is referred to as the “closed” state of the self-close valve.

Superabsorbent polymers can absorb and retain large volumes of water or other aqueous solutions. In some examples, SAP can be made from chemically modified starch and cellulose and other polymers, such as polyvinyl alcohol) PVA, poly(ethylene oxide) PEG, which are hydrophilic and have a high affinity for water. In some examples, superabsorbent polymers can be made of partially neutralized, lightly cross-linked poly(acrylic acid), which has a good performance versus cost ratio. The polymers can be manufactured at low solids levels, then dried and milled into granular white solids, hi water, the white solids swell to a rubbery gel that in some cases can include water up to 99% by weight.

Referring to FIG. 7A, a self-close valve 170 can include a channel 164 that has an enlarged portion 172 to accommodate the superabsorbent polymers 162 so that the superabsorbent polymers 162 do not restrict flow of fluid before expansion of the SAP 162. To fabricate the self-close valve 170, an adhesive can be applied to the inner wails of the enlarged portion 172, the SAP 162 in powder form is then pushed into the channel 164 so that the SAP 162 powder adheres to the inner wall at the enlarged portion 172.

Referring to FIG. 7B, as the fluid flows past the superabsorbent polymers 162, the superabsorbent polymers 162 absorb a portion of the fluid and expands in volume, blocking the channel 164, preventing further flow of the fluid past the expanded polymers 162.

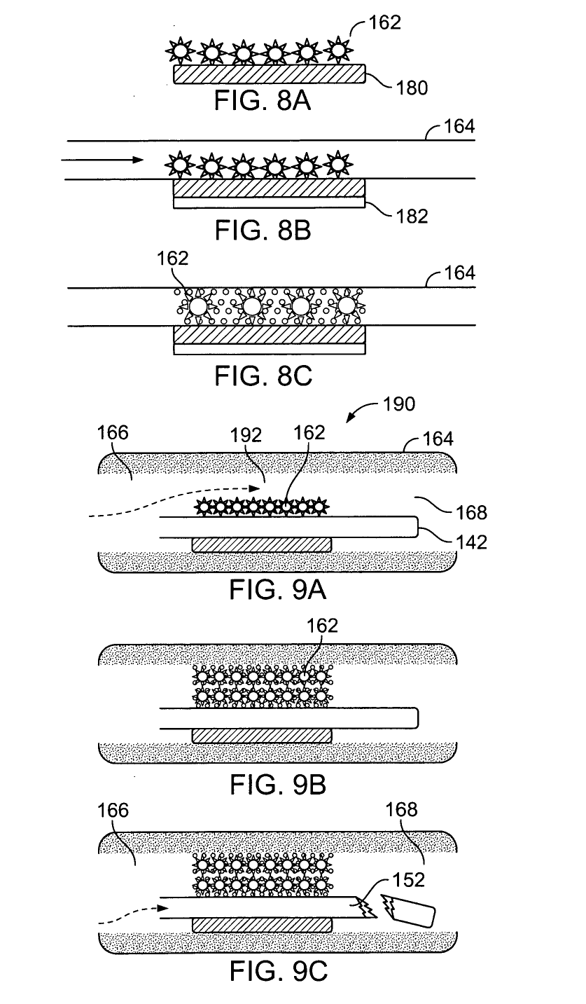

Referring to FIGS. 8A and 8B, superabsorbent polymers 162 can be attached to a wire 180, then placed into a channel 164. The channel 164 can have a recessed region 182 in which, an adhesive is applied to secure the wire 180 at a predefined location.

Referring to FIG. 8C, as the fluid flows past the superabsorbent polymers 162, the polymers 162 absorb a portion of the fluid and expands in volume, blocking the channel 164, preventing further flow of the fluid past the expanded polymers 162.

A self-close valve can be constructed by coating a wire with SAP, then placing the coated wire into a channel or tube. A self-close valve for use in a planar fluidic device can be constructed by coating a planar substrate with SAP, then placing the coated substrate into a planar channel, in the planar fluidic device.

Referring to FIGS. 9A to 9C, an on-off-on valve 190 can be fabricated by using a glass capillary 142 and SAP 162 that are positioned outside of and adjacent to the capillary 142. The capillary 142 and the SAP 162 are both positioned in a channel 164 having a first region 166 and a second region 168. Using the glass capillary 142 and the SAP is similar to using a combination of a broken open valve and a self-close valve. The on-off-on valve 190 enables a user to control the flow of fluids through a particular location in the channel by allowing, then blocking, and then allowing fluids to pass through the particular location.

Referring to FIG. 9A, initially, the SAP 162 has a smaller volume and does not block the channel, allowing a fluid to flow between, the first and second regions 166 and 168.

Referring to FIG. 9B, as the fluid passes, a portion of the fluid is absorbed by the SAP 162, causing the SAP 162 to increase in volume, blocking further flow of the fluid between the first and second regions 166 and 168.

Referring to FIG. 9C, when an external force is applied to break the glass capillary 142, a passage 152 is generated to allow the fluid to flow between the first and second regions 166 and 168.

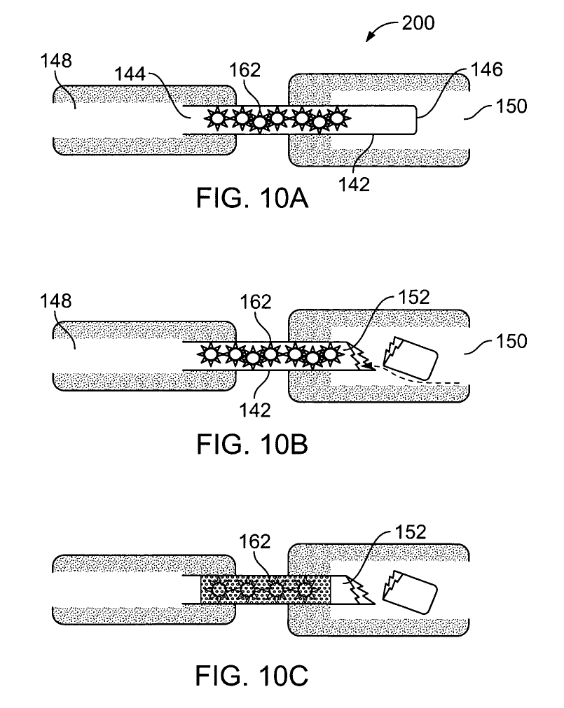

Referring to FIGS. 10A to 10C, an off-on-off valve 200 can be fabricated by using a glass capillary 142 and SAP 162 that are positioned inside the capillary 142. The capillary 142 has an open end 144 and a closed end 146. The open end 144 is positioned in a first channel 148, and the closed end 146 is positioned in a second channel 150. The glass capillary 142 and the SAP 162 perform functions similar to a combination of a broken open valve and a self-close valve. The off-on-off valve 200 enables a user to control, the flow of fluids through a particular location in the channel by blocking, then allowing, and then blocking fluids from passing through the particular location.

Referring to FIG. 10A, when the glass capillary 142 is intact, the first and second channels 148 and 150 are not connected.

Referring to FIG. 10B, when an external force is applied to break the glass capillary 142, a passage 152 is formed, allowing fluid to flow between the channels 148 and 150. The SAP 162 initially has a smaller volume and does not block the flow of fluid in the passage 152.

Referring to FIG. 10C, as the fluid flows through the passage 152, a portion of the fluid is absorbed by the SAP 162, causing the SAP to increase in volume and block the passage 152, preventing further flow of the fluid through the passage 152.

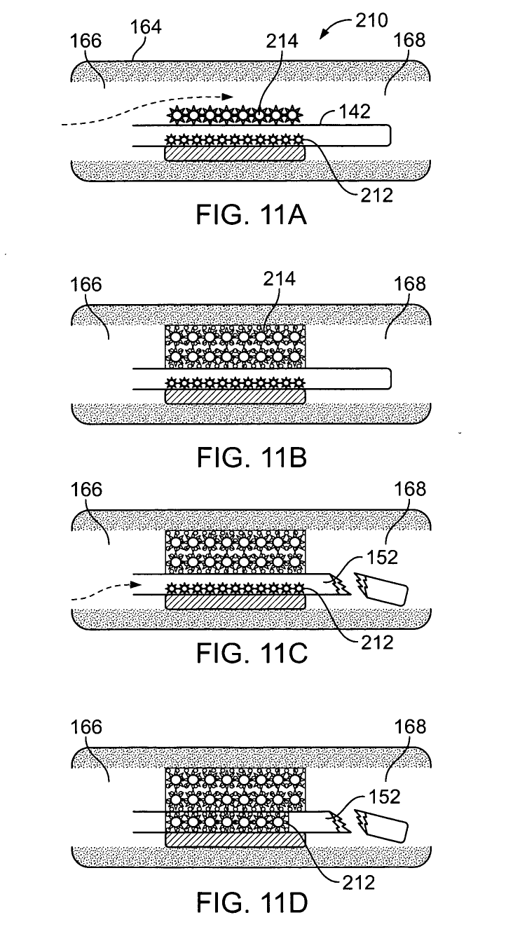

Referring to FIGS. 11A to 11D, an on-off-on-off valve can be fabricated by using a glass capillary 142, SAP 212 that are positioned inside the capillary 142, and SAP 214 that are positioned outside of the capillary 142. The glass capillary 142, the SAP 212, and the SAP 214 are placed in a channel 164. The glass capillary 142, the SAP 212, and the SAP 214 perform functions similar to a combination of a broken open valve and two self-close valves. The on-off-on-off valve 210 enables a user to control the flow of fluids through a particular location in the channel by allowing, then blocking, then allowing, and then blocking fluids from passing through the particular location.

Referring to FIG. 11A, initially, the SAP 214 has a smaller volume and allows a fluid to flow between a first region 166 and a second region 168 of the channel 164.

Referring to FIG. 11B, as fluid passes, a portion of the fluid is absorbed by the SAP 214, causing the SAP 214 to increase in volume, blocking further flow of the fluid between the first and second regions 166 and 168.

Referring to FIG. 11C, when an external force is applied to break the glass capillary 142, a passage 152 is formed to allow fluids to flow between the first and second regions 166 and 168.

Referring to FIG. 11D, as the fluid flows pass the SAP 212, a portion of the fluid is absorbed by the SAP 212, causing the SAP 212 to increase in volume and block the passage 152, preventing further flow of fluids through the passage 152.

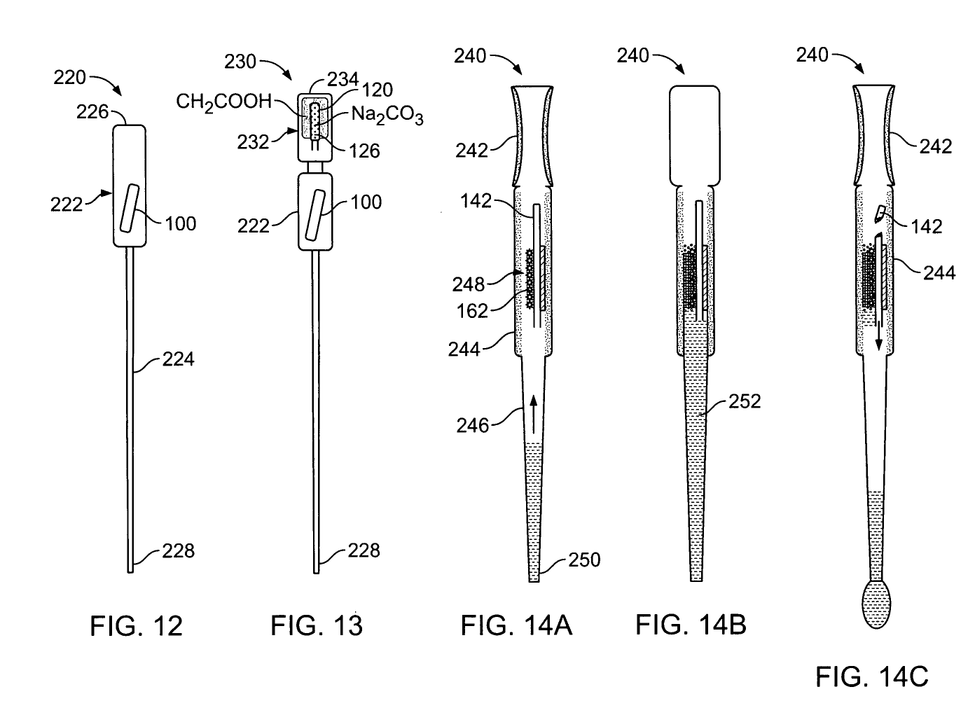

Referring to FIG. 12, a metering pipette 220 for drawing a predetermined amount of fluid can be constructed by using a vacuum pump 222 coupled to a pipette tube 224. The vacuum pump 222 includes a vacuum glass capillary 100 that is placed in a pipette bulb 226. To use the metering pipette 220, the glass capillary 100 is broken to generate a suction force that draws a fluid into the pipette tube 224.

When a batch of metering pipettes 220 are manufactured, the sizes of the bulb 226 and the glass capillary 100 can be made to be the same. The bulb 226 and the glass capillary 100 are designed so that when the user presses the bulb 226 to break the glass capillary 100, the amount of deformation imparted on the bulb 226 that is required to cause the glass capillary 100 to be broken is substantially the same for all the metering pipettes 220. This way, a user can use the metering pipette 220 to quickly draw in a predetermined amount of fluid without monitoring the fluid level in the stem 224.

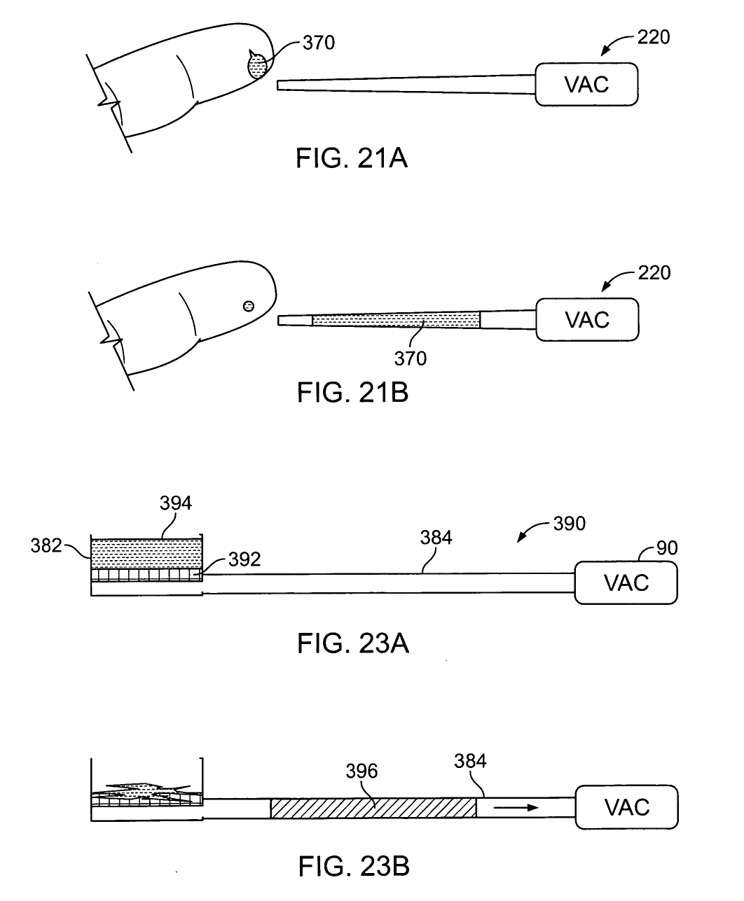

For example, referring to FIGS. 21A and 21B, a metering pipette 220 can be used to quickly sample a predetermined amount of blood 370 from a patient.

Referring to FIG. 13, another example of a metering pipette 230 includes a vacuum pump 222 and a gas pump 232. The vacuum pump 222 is similar to that shown in FIG. 12. The gas pump 232 includes a glass capillary 120 filled with Na₂CO₃ and placed in a pipette bulb 234 containing CH₂COOH. When the glass capillary 120 is broken, Na₂CO₃ interacts with CH₂COOH to generate CO₂, increasing the gas pressure in the bulb 234. The vacuum pump 222 allows the user to quickly draw a predetermined amount of a fluid into the pipette 230. The gas pump 232 allows the user to dispense the fluid out of the pipette 230.

An advantage of using the gas pump 232 is that the fluid in the tube 228 can be dispensed over a controlled period of time as the CO₂ gas is generated from the reaction between Na₂CO₃ and CH₂COOH. This way, the user does not have to carefully monitor the output flow of the fluid when dispensing the fluid.

Referring to FIG. 14A, another example of a metering pipette 240 includes a bulb 242, a middle section 244, and a pipette tube 246. The middle section 244 is constructed of a deformable material. An on-off-on valve 248 is positioned in the middle section 244. The on-off-on valve 248 includes a glass capillary 142 and SAP 162 positioned outside of the capillary 142, similar to the device shown in FIGS. 9A to 9C.

Referring to FIG. 14A, to use the pipette 240, the user squeezes and releases the bulb 242 to draw a fluid into the tube 246 and the middle section 244.

Referring to FIG, 14B, when the fluid reaches the middle section 244 and comes into contact with the SAP 162, a portion of the fluid is absorbed by the SAP 162, causing the SAP 162 to expand in volume and block passage of the fluid beyond the SAP 162. This way, a predetermined amount of fluid is drawn into the pipette 240.

Referring to FIG. 14C, to dispense the fluid from the pipette 240, the user presses the middle section 244 (which is made of deformable material) to break the glass capillary 142, forming a passage through the broken capillary 142. The user then squeezes the bulb 242 to force the fluid out of the pipette 240.

When a batch of pipettes 240 are manufactured, the size of the tube 246 and the middle section 244, and the position of the on-off-on valves 248 within the middle section 244 are the same, so that users can use the pipettes 240 to quickly draw in substantially the same amounts of fluids without closely monitoring the levels of liquids in the pipettes 240.

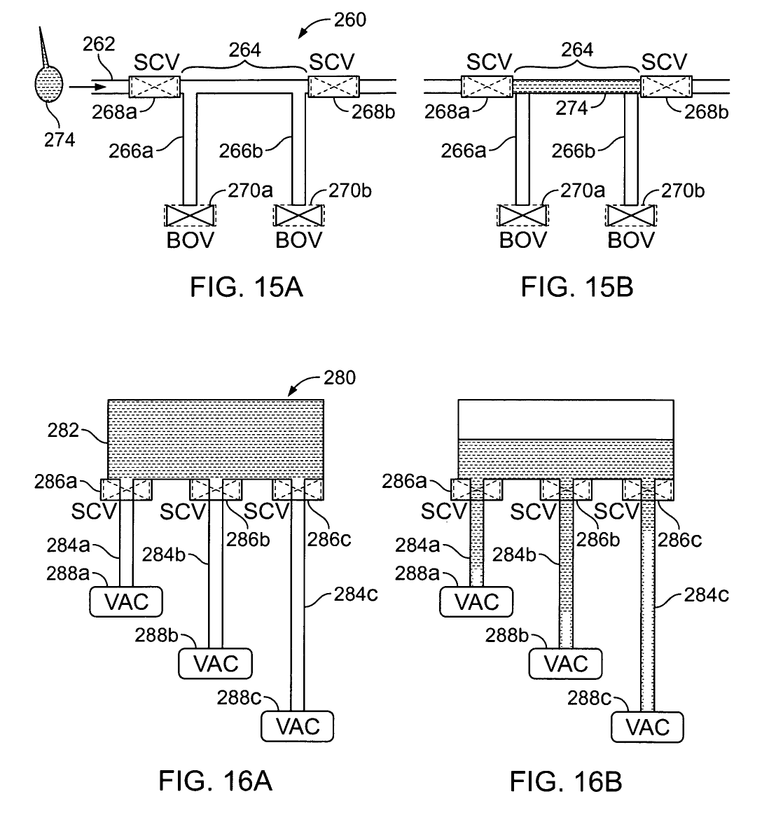

Referring to FIG. 15A, a metering device 260 for collecting a predetermined amount of fluid includes a glass capillary 262 having two branches 266a and 266b, two self-close valves 268a and 268b, and two broken open valves 270a and 270b. Each of the self-close valves 268a and 268b has SAP that expands upon absorption of fluids. Initially, the self-close valves 268a and 268b are in the open state, and the broken open valves 270a and 270b are in the closed state. The self-close valves 268a and 268b can be similar to those shown in FIGS. 6A to 8C. The broken open valves 270a and 270b can be similar to those shown in FIGS. 5A and 5B.

In operation, a fluid 274 is drawn into the capillary 262 due to a capillary force, and flows past the self-close valves 268a and 268b. Referring to FIG. 15B, as the fluid 274 flows pass the self-close valves 268a and 268b, a portion of the fluid 274 is absorbed by the SAP in the self-close valves 268a and 268b, causing the self-close valves 268a and 268b to change to the closed state, blocking further passage of the fluid 274. This results in the fluid 274 occupying a segment 264 of the capillary between the self-close valves 268a and 268b.

The fluid 274 can be moved from the segment 264 to other locations through the branch 266a or 266b by changing the broken open valves 270a and 270b from the closed slate to the open state, and applying a suction force or a push force to move the fluid 274.

An advantage of the metering device 260 is that it can quickly sample a predetermined volume of fluid without careful monitor by the user. Because the capillaries of the metering device 260 have small diameters, the metering device 260 is useful in precisely sampling small amounts of fluid.

Referring to FIG. 16A, a metering device 280 that can obtain three different amounts of fluids from a sample well 282 includes three capillaries 284a, 284b, and 284c. Each capillary has a self-close valve (e.g., 286a, 286b, or 286c) at one end and a vacuum valve (e.g., 288a, 288b, or 288c) at the other end. Each vacuum pump has a vacuum glass capillary. Initially, the self-close valves are in the open state.

Referring to FIG. 16B, when the user breaks the vacuum glass capillary in the vacuum pumps 288a, a suction force is generated to draw a predefined amount of liquid into the capillary 284a. As the fluid passes the self-close valve 286a, the SAP in the self-close valve 286a expands, causing the self-close valve 286a to enter the closed state, preventing further movement of the fluid through the self-close valve 286a. Similarly, predefined amounts of fluid can be drawn into the capillaries 284b and 284c by breaking the vacuum capillaries in the vacuum pumps 288b and 288c. The amounts of fluid drawn into the capillaries 284a to 284c are determined by the volumes of the capillaries in the vacuum pumps 288a to 288c, which can be the same or different.

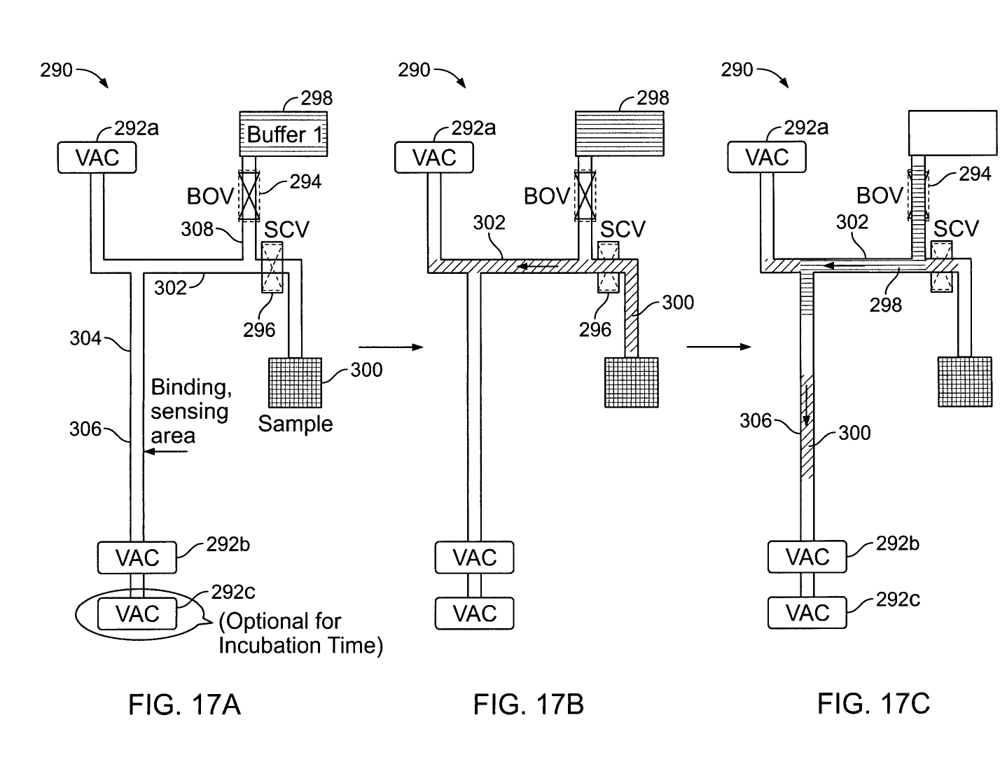

Referring to FIG. 17A, a device 290 for use in a two-step assay that requires rapid binding of reagents followed by washing with a buffer can be fabricated using a combination of vacuum pumps, a broken-open valve, and a self-close valve. A channel 302 has one end coupled to a sample well containing a sample 300 through a self-close valve 296, and another end coupled to a first vacuum pump 292a. The channel 302 is connected to a channel 308, which is coupled to a buffer 298 through a broken-open valve 294. The channel 302 is also connected to a channel 304, which is coupled to a second vacuum pump 292b and a third vacuum pump 292c. The channel 304 includes a binding and/or sensing area 306 that includes reagents for binding or sensing compounds in the sample 300.

The device 290 is operated in a way such that the sample 300 is drawn towards the binding and sensing area 306 to cause a reaction to occur, then the buffer 298 is drawn towards the binding and sensing area 306 to wash the binding and sensing area 306.

Referring to FIG. 17B, the vacuum pump 292a is activated to generate a suction force that draws the sample 300 towards the vacuum pump 292a and into the section of the channel 302 between the vacuum pump 292a and the self-close valve 296. As the sample 300 flows past the self-close valve 296, a portion of the sample is absorbed by the SAP in the self-close valve 296, causing the self-close valve 296 to enter the closed state.

Referring to FIG. 17C, the broken-open valve 294 is activated to cause the valve 294 to change to the open state. The vacuum pump 292b is activated to generate a suction force that draws both the sample 300 and the buffer 298 towards the vacuum pump 292b. The vacuum pumps 292a and 292b are designed such that after the pumps are activated, the sample 300 will stop at the binding and sensing area 306. After a period of time, the vacuum pump 292c is activated to move the sample 300 out of the area 306, and cause the buffer 298 to flow through and wash the area 306.

The example above provides incubation time that allows the compounds in the sample 300 to react with the reagents in the binding and sensing area 306 before the area 306 is washed by the buffer 290. If the reactions at the area 306 is fast and incubation time is not necessary, then the vacuum pump 292b can be made larger and the vacuum pump 292c can be omitted. When the vacuum pump 292b is activated, the sample rapidly flows pass the binding and sensing area 306, followed by washing by the buffer 298.

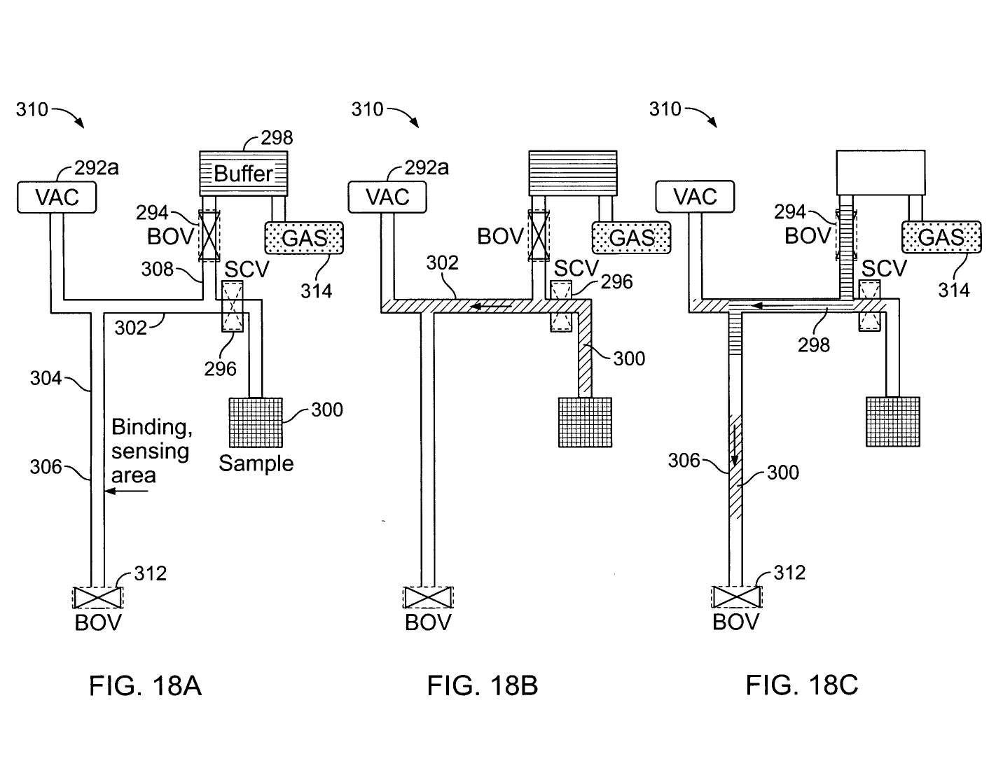

Referring to FIG. 18A, a device 310 for use in a two-step assay that requires slow binding of reagents followed by washing with a buffer can be lubricated using a combination of a vacuum pump, broken-open valves, a self-close valve, and a gas pump. The device 310, similar to the device 290, has a channel 302 connected to two channels 304 and 308. The channel 302 is coupled to a sample 300 through a self-close valve 296. The channel 308 is coupled to a buffer 298 through a broken-open valve 294. The channel 304 includes a binding and sensing area 306. One end of the channel 304 is coupled to a broken-open valve 312. A gas pump 314 is coupled to the buffer 298.

The difference between the device 310 and the device 290 is that, in device 310, rather than using the vacuum pump 292b to draw the sample 300 and buffer 298 towards the binding and sensing area 306, the gas pump 314 is used to push the sample 300 and the buffer 298 towards the area 306.

Referring to FIG. 18B, to perform the two-step assay, the vacuum pump 292a is activated to draw the sample 300 into the channel. The self-close valve 296 enters a closed state after the sample flows pass the valve 296.

Referring to FIG. 18C, the broken-open valves 294 and 312 are activated to cause the valves to change to the open state. The gas pump 314 is activated to generate gas over a period of time, pushing the sample 300 and the buffer 298 through the binding and sensing area 306. Because the gas pump 314 generates gas over a period time (the reaction between compounds that generate gas takes a certain amount of time to complete), the sample 300 can pass the binding and sensing area 306 slowly, allowing slow binding reactions to occur.

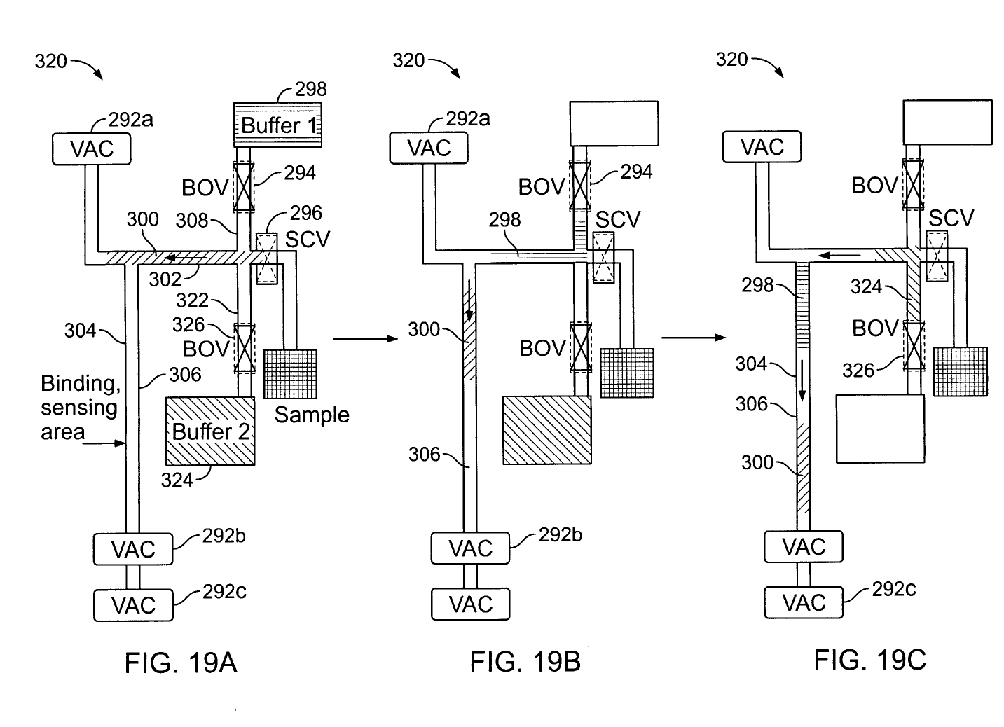

Referring to FIG. 19A, a device 320 for use in a three-step assay that requires rapid binding of reagents followed by washing with two buffers can be constructed by adding a second buffer 324, and a channel 322 to the structure show in FIG. 17A. To perform the multi-step assay, the vacuum pump 292a is activated to cause the sample 300 to How to the channel 302. As the sample 300 flows past the self-close valve 296, the valve 296 changes to a closed state.

Referring to FIG. 19B, the broken-open valve 294 is activated so that if changes to an open state, and the vacuum pump 292b is activated to cause the sample 300 and the first buffer 298 to be drawn toward the binding and sensing area 306.

Referring to FIG. 19C, the broken-open valve 326 is activated so that it changes to an open state, and the vacuum pump 292c is activated to cause the sample 300, the first buffer 298, and the second buffer 324 to be drawn towards the binding and sensing area 306. This way, the reaction at the area 306 can be washed by two different buffers.

A device for use in assays that require more than three steps can be constructed by coupling additional buffers or samples, and adding a corresponding number of vacuum pumps to the end of the channel 304.

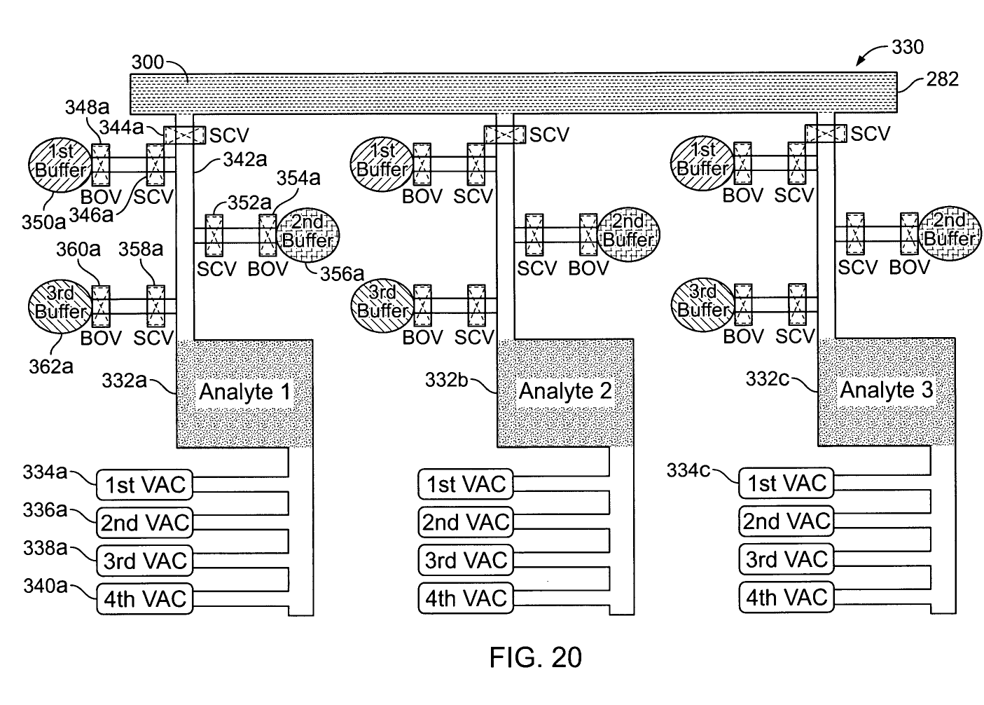

Referring to FIG. 20, a module 330 can be constructed to perform multiplex analyte assay. The module 330 includes a sample well 282 for holding a sample 300 and three chambers 332a, 332b, and 332c, each containing an analyte for binding and sensing compounds in the sample 300. Below is a description of the components used to perform an assay concerning the first analyte in the chamber 332a.

The chamber 332a is coupled to the sample well 282 through a channel 342a and a self-close valve 344a. The channel 342a is coupled to a first buffer 350a through a self-close valve 346a and a broken-open valve 348a. The channel 342a is coupled to a second buffer 356a through a self-close valve 352a and a broken-open valve 354a. The channel 342a is coupled to a third buffer 362a through a self-close valve 358a and a broken-open valve 360a. The chamber 332a is also connected to vacuum pumps 334a, 336a, 338a, and 340a.

To perform the assay, the vacuum pump 334a is activated to draw the sample 300 towards the chamber 332a to allow the compounds in the sample 300 to react with the first analyte in the chamber 332a. After a certain amount of the sample flows through the self-close valve 344a, the valve 344a changes to the closed state. The first buffer 350a is flushed through the chamber 332a by activating the broken-open valve 348a (to change the valve to the open state) and the second vacuum pump 336a. After a certain amount of the first buffer 350a flows past the self-close valve 346a, the valve 346a changes to a closed state.

The second buffer 356a is flushed through the chamber 332a by activating the broken-open valve 354a (to change the valve to the open state) and the third vacuum pump 338a. After a certain amount of the second buffer 356a flows past the self-close valve 352a, the valve 352a changes to a closed state.

In a similar manner, the third buffer 362a is flushed through the chamber 332a by activating the broken-open valve 360a (to change the valve to the open state) and the fourth vacuum pump 340a. After a certain amount of the third buffer 362a flows past the self-close valve 358a, the valve 358a changes to a closed state.

The assays concerning the second and third analytes in the chambers 332b and 332c can be performed similar to the manner that the assay concerning the first analyte in the chamber 332a is performed. The assays concerning the first, second, and third analytes in the chambers 332a, 332b, and 332c can be performed simultaneously.

The following are applications of the vacuum pumps and gas pumps in performing biological assays.

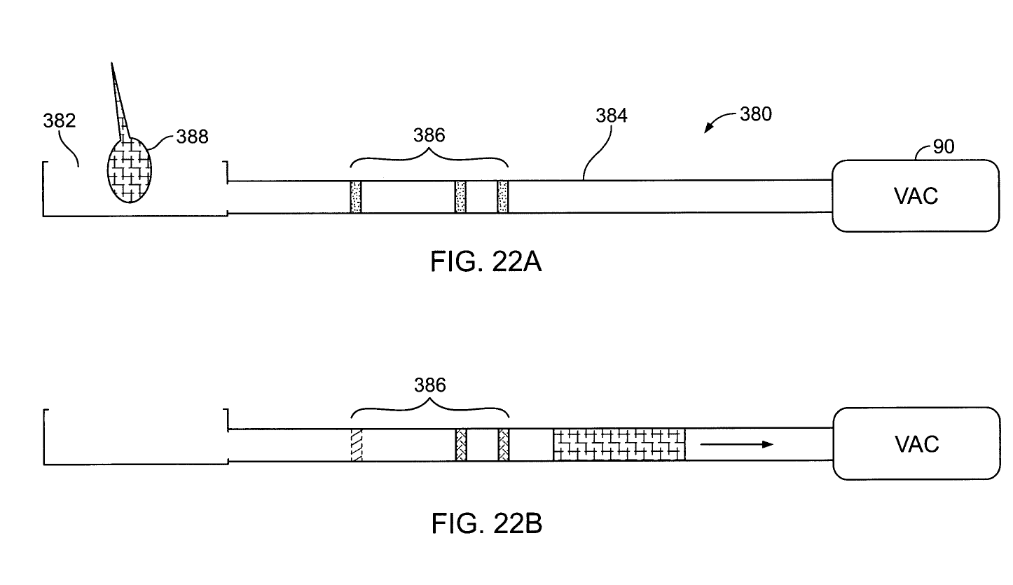

FIGS. 22A and 22B show a device 380 for performing rapid reaction colorimetric assay. The device 380 includes a channel 384 coupled to a sample well 382 at one end and coupled to a vacuum pump 90 at the other end. The sample well 382 can hold a sample fluid 388, such as blood or urine. The channel 384 includes a testing area 386 having test lines that change color upon detection of certain compounds. The vacuum pump 90 when activated can quickly draw the fluid in the sample well 382 through the testing area 386. By reading the color of the test lines, a user can quickly determine the existence or non-existence of certain compounds in the fluid.

FIGS. 23A and 23B show a device 390 for sampling a filtered fluid. The device 390 includes a channel 384 that has one end coupled to a sample well 382 and another end coupled to a vacuum pump 90. A filter membrane 392 is placed in the sample well 382. The vacuum pump 90 when activated can quickly draw a fluid 394 (e.g., blood) in the sample well 382 through the filter membrane 392, producing a filtered fluid 396 (e.g., plasma) that is drawn into the channel 384.

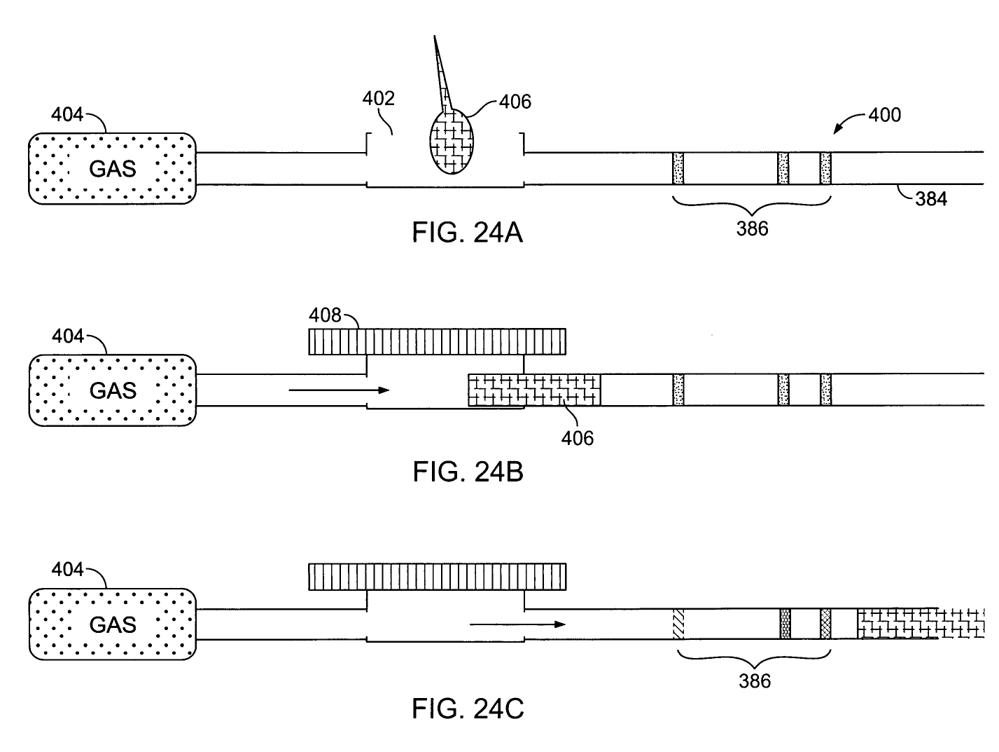

FIGS. 24A to 24C show a device 400 for performing a slow colorimetric assay. Referring to FIG. 24A, the device 400 includes a sample well 402 coupled between a gas pump 404 and a channel 384. The channel 384 has a test area 386 having test lines that change color upon detection of certain compounds. To use the device 400, a sample fluid 406 is placed in the sample well 402. Referring to FIG. 24B, a sealing tape 408 seals the opening of the sample well 402. Referring to FIG. 24C, the gas pump 404 is activated to generate gas that pushes the sample fluid 406 through the test area 386. Because the gas pump 404 generates gas over a period of time, the sample fluid 406 travels through the test area over a period of time, allowing a slow colorimetric assay to be performed.

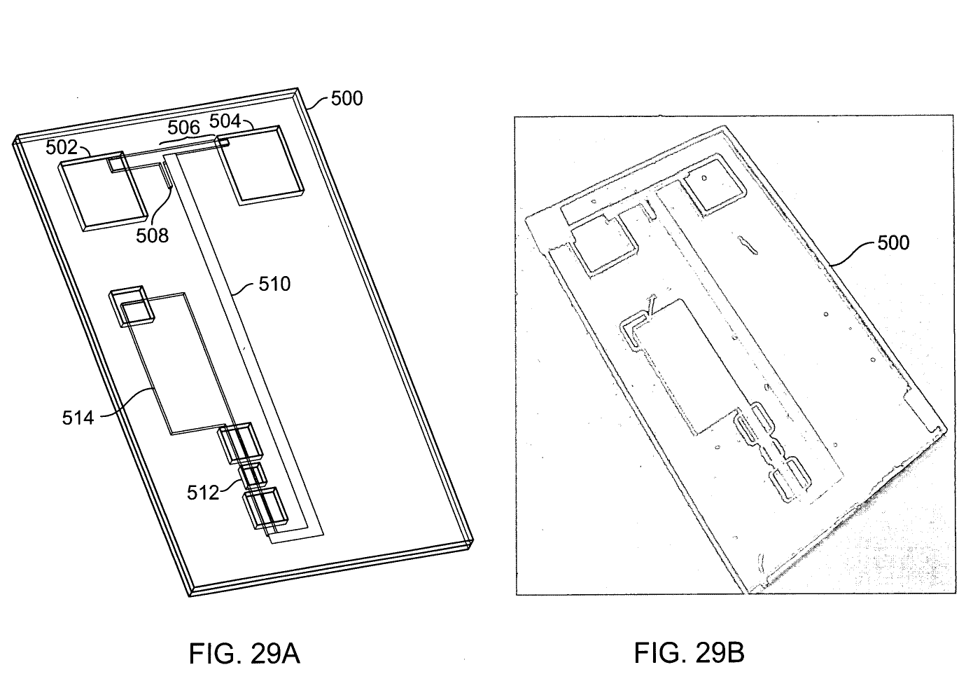

FIGS. 29A and 29B show a diagram and a photograph, respectively, of an example of a device 500 for performing an immunoassay. The device 500 includes a blood sample well 502, a washing buffer well 504, a metering zone 508 with labeled antibody (Ab*), a self-close valve 508, a diagnostic zone 510 having an antibody array, a broken open valve 512, and a waste well 514. The main body of the device 500 can be made of, e.g., glass or plastic. The self-close valve 508 can be filled with SAP that, upon contact with a fluid, expands to close off the capillary adjacent to the self-close valve 508.

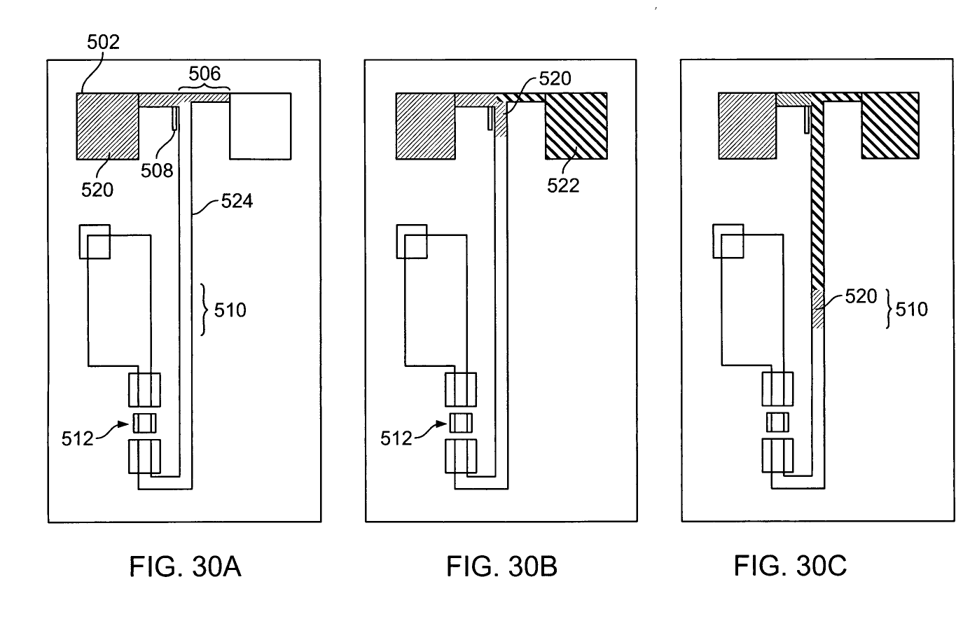

Referring to FIG. 30A, an immunoassay can be performed by placing a blood sample 520 in the sample well 502. Some of the blood is drawn to the metering zone 508 by capillary force and mixed with the labeled antibody (Ab*). Some of the blood is absorbed by the SAP in the self-close valve 508, causing the SAP to expand in volume to block the capillary and prevent additional blood from entering the metering zone 508. This way, a controlled amount of blood sample can be obtained in the metering zone 508. Initially, the broken open valve 512 is closed, so that the blood enters the capillary of the metering zone 506 and does not enter the capillary 524 that is coupled to the diagnostic zone 510.

Referring to FIG. 308, after about 30 to 60 seconds to allow the blood sample 520 to have sufficient time to mix with the labeled antibody (Ab*), a washing buffer 522 is loaded to the washing buffer well 504. The broken open valve 512 is activated and switches to an open state. The metered blood sample 520 and the washing buffer 522 are drawn to the capillary 510 due to capillary force.

Referring to FIG. 30C, the blood sample 520 enters the diagnostic zone 510. If the blood sample 520 has one or more particular types of antigen (Ag) that match the antibody (Ab) in the diagnostic zone 510, binding of antigen (Ag), antibody (Ab), and the labeled antibody (Ab*) will occur. Afterwards, the blood sample 520 and unbound molecules are washed away by the washing buffer 522. The labeled antibody (Ab*) bound to the diagnostic zone 510 can then be read by an optical reader.

The device 500 provides a simple way to determine whether the blood sample has certain types of antigen, such as cardiac markers, myoglobin, CK-MB, and troponin I, heart failure markers B-type natriuretic peptide (BNP), inflammatory marker C-reactive protein (CRP), etc. The device 500 can be used for qualitative, semi-quantitative, and quantitative determinations of one or multiple analytes in a single test format. The device 500 can be used to perform, e.g., fluorescence-linked immunosorbent assay (FLISA), enzyme-linked immunosorbent assay (ELISA), sol particle, and other assay formats, and is suitable for simultaneous multiple analyte assays,

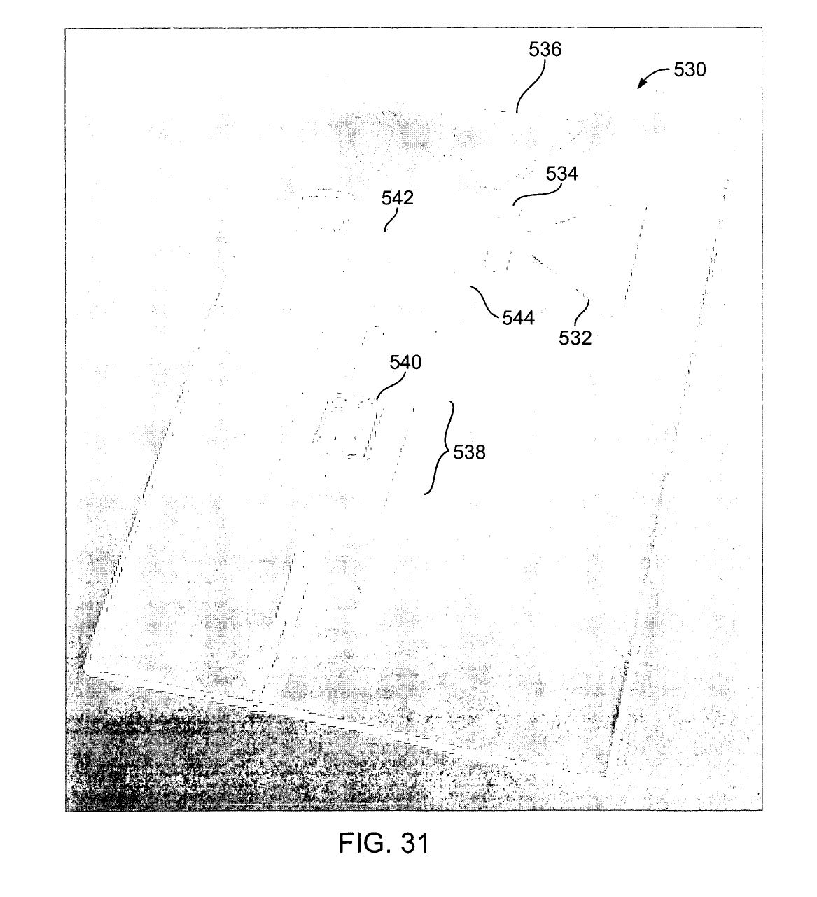

FIG. 31 is a photograph of another example of a device 530 for performing an immunoassay. The device 530 includes a blood sample well 532, a self-close valve 534, a washing buffer well 536, a diagnostic zone 538, a broken open valve 540, and a waste zone 542. Initially, a blood sample is loaded to the blood, sample well 532. The blood is drawn to a capillary 544 coupled to the diagnostic zone 538 by capillary force. The blood sample well 532 includes a blood cell removal membrane, so that only blood plasma passes the membrane and enters the capillary 544. A portion of the blood plasma is absorbed by the SAP in the self close valve 534, causing the valve 534 to enter a closed state, preventing additional blood plasma from entering the capillary 544. This allows a controlled volume of blood plasma to be obtained.

A washing buffer is loaded to the washing buffer zone 536. The broken open, valve 540 is activated and switches to an open state. The blood plasma and the washing buffer are drawn to the diagnostic zone 538 due to capillary force. The diagnostic zone 538 has an array of antibody molecules. If the blood plasma has one or more particular types of antigen that matches one or more of the antibody in the diagnostic zone 538, binding of antigen and antibody will occur. The blood plasma and the non-binding molecules are washed away by the washing buffer. The bound molecules in the diagnostic zone 538 can be read by an optical sensor.

The device 530 provides a simple way to determine whether the blood sample has certain types of antigen, such as cardiac markers, myoglobin, CK-MB, and troponin I, heart failure markers B-type natriuretic peptide (BNP), inflammatory marker C-reactive protein (CRP), etc. The device 530 can be used for qualitative, semi-quantitative, and quantitative determinations of one or multiple analytes in a single test format. The device 530 can be used to perform fluorescence-linked immunosorbent assay (FLISA), enzyme-linked immunosorbent assay (ELISA), sol particle and other assay formats, and is suitable for simultaneous multiple analyte assays.

Although some examples have been discussed above, other implementations and applications are also within the scope of the following claims. For example, in the vacuum pump 90 of FIGS. 1A and 1B, the container 100 can container a low pressure region instead of a vacuum region. As long as the gas pressure inside the container 100 is lower than the gas pressure outside of the container 100, when the container 100 breaks, the pressure in the region 106 outside of the container 100 will drop, generating a suction force that draws fluids in a direction towards the container 100. The glass capillaries described above can be replaced by capillaries made of other brittle materials, such as brittle plastic, quartz, and ceramic.

What is claimed is:

1. A method comprising
controlling a flow of a fluid in a channel, including breaking a first container to generate a pressure difference in the channel to cause the fluid to move in the channel, the first container being made of a brittle material, wherein the first container (a) defines a space within the first container having a gas pressure that is different from the gas pressure outside of the first container, or (b) includes a first material that is separated from a second material prior to the breaking of “the first container, the first and second materials selected to generate gas upon interaction of the first and second materials.

2. The method of claim 1 wherein the space in the first container has a pressure higher than the pressure outside of the first container, and controlling the flow of the fluid comprises pushing the fluid in the channel away from the broken first container.

3. The method of claim 1 wherein the space in the first container has a pressure lower than the pressure outside of the first container, and controlling the flow of the fluid comprises attracting the fluid in the channel towards the broken first container,

4. The method of claim 3 wherein the fluid comprises blood, and controlling the flow of the blood comprises passing the blood through a filter to block blood cells and to allow blood plasma to pass the filter and enter the channel.

5. The method of claim 3, further comprising performing a colorimetric assay as the fluid flows in the channel.

6. The method of claim 3 further comprising a second container that (a) defines a space within the second container having a gas pressure that is higher than the gas pressure outside of the second container, or (b) includes a third material that is separated from a fourth material prior to the breaking of the second container, the third and fourth materials selected to generate gas upon interaction of the third and fourth materials.

7. The method of claim 6 wherein controlling the flow of the fluid comprises pushing the fluid in the channel away from the second container.

8. The method of claim 3 wherein controlling the flow of the fluid comprises preventing movement of additional fluid through the channel in a certain direction by using a fluid absorbing material that expands in volume upon absorption of a portion of the fluid.

9. A fluidic device comprising:
a channel; and
a first container that generates a pressure difference in the channel when the first container is broken, the first container being made of a brittle material, wherein the first container (a) defines a space within the first container having a gas pressure that is different from the gas pressure outside of the first container, or (b) includes a first material that is separated from a second material prior to the breaking of the first container, the first and second materials selected to generate gas upon interaction of the first and second materials.

10. The fluidic device of claim 9, further comprising a second container that (a) defines a space within the second container having a gas pressure that is different from the gas pressure outside of the second container, or (b) includes a third material that is separated from a fourth material prior to the breaking of the second container, the third and fourth materials selected to generate gas upon interaction of the third and fourth materials.

11. The fluidic device of claim 10 wherein the space in the first container has a pressure higher than the pressure outside of the first container, and the space in the second container has a pressure lower than the pressure outside of the second container.

12. The fluidic device of claim 10 wherein the space in the first container has a pressure lower than the pressure outside of the first closed container, and the second container includes a first material that is separated from a second material prior to the breaking of the second container, the first and second materials selected to generate gas upon interaction of the first and second materials.

13. The fluidic device of claim 12, further comprising a filter membrane to block blood cells and to allow blood plasma to pass.

14. The fluidic device of claim 9 wherein the brittle material comprises at least one of quartz, glass, ceramic, plastic, and a composite of two or more of quartz, glass, ceramic, and plastic.

15. The fluidic device of claim 9 wherein the channel is defined by a wall made of a flexible material.

16. The fluidic device of claim 9 wherein the first container includes a material that generates gas when heated.

17. The fluidic device of claim 9 wherein the first container includes a material that sublimes from a solid state to a gas state when heated.

18. A method comprising:
providing a plurality of pipettes to enable sampling of predetermined amounts of fluids, each pipette including
a channel, and
a container that generates a pressure difference in the channel when, the container is broken, the container being made of a brittle material, the container defining a space within the container having a gas pressure that is less than the gas pressure outside of the container, wherein breaking the container generates a predetermined amount of pressure difference in the channel to cause a predetermined amount of fluid to be drawn into the channel.

19. The method of claim 18 wherein each pipette includes a second container that generates a pressure difference in the channel when the second container is broken, the second container (a) defining a space within the second container having a gas pressure mat is higher than the gas pressure in the channel, or (b) including a first material that is separated from a second material prior to the breaking of the second container, the first and second materials selected to generate gas upon interaction of the first and second materials.

20. The method of claim 18 wherein the brittle material comprises at least one of quartz, glass, ceramic, plastic, and a composite of two or more of quartz, glass, ceramic, and plastic.
